(19) United States
(12) Patent Application Publication
(10) Pub. No.: US 2013/0014765 A1
(43) Pub. Date: Jan. 17, 2013

(54) TONGUE AND MANDIBULAR ADVANCEMENT APPLIANCE

(71) Applicant: Thomas E. Meade, Albuquerque, NM (US)

(72) Inventor: Thomas E. Meade, Albuquerque, NM (US)

(21) Appl. No.: 13/184,500

(22) Filed: Jul. 16, 2011

(51) Int. Cl. A61F 5/56

(52) U.S. Cl. 128/848

(57) ABSTRACT

A tongue and mandibular advancement device for pulling the jaw forward and keeping the tongue in an extended position includes upper and lower inserts conforming to upper and lower teeth respectively. A hook support is located on both sides of the upper insert at a forward position and a plurality of hook supports are located at a rearward position of both sides of the lower insert. Tension coil springs are fitted to each of the upper insert hook supports and to one of the plurality of lower hook supports. The plurality of lower insert hook supports provides discrete levels of tension force of the coil spring on each side of the appliance. A plastic tube may be fitted over the coil springs to prevent injury to the inside of a user's mouth. The upper and lower inserts each have a magnet that attracts a magnetizable member on or in a user's tongue to keep the tongue at an extended position.

### FIELD

This invention is related to oral appliances for preventing or alleviating snoring and sleep apnea. The appliance is a removable tongue and mandibular advancement device that uses coil springs to pull the lower jaw forward during use and to keep the tongue of a user in an extended position.

### BACKGROUND

It is known in the art that an oral appliance that serves to move the mandible forward reduces sleep apnea and snoring of a user. A variety of appliances are available that are removable and advance the mandible. Some of these appliances are adjustable to provide different mandible advancement forces.

One such appliance issued to Frantz et al on Aug. 29, 2000, U.S. Pat. No. 6,109,265, uses elastic bands hooked between upper and lower trays to pull the mandible forward. The elastic urethane bands are interchangeable with other bands having varying lengths and/or elasticity to provide different advancement forces on the mandible. This appliance uses rubber bands, which may tend to break with use, and requires new rubber bands to be applied to change the tension.

Other such appliances are disclosed in U.S. Pat. No. 4,505,672 to Kurz, U.S. Pat. No. 5,775,219 to Thronton, U.S. Pat. No. 6,450,167 to David et al., U.S. Pat. No. 5,947,724 to Frantz et al., U.S. Pat. No. 6,729,335 to Halstrom, and U.S. Pat. Nos. 5,467,783, 5,682,903, and 6,055,986 to Meade.

### SUMMARY

The present invention is a removable, adjustable oral appliance for alleviating snoring and sleep apnea in a user. The appliance has tension springs fitted between hook supports on both sides of an upper teeth conforming insert and extending in tension to one of a plurality of hook supports on the teeth conforming lower insert. These springs provide discrete levels of tension force to the lower jaw to advance the jaw forward. The user's tongue may kept at an extended position by placing of the magnets on the upper and lower inserts that attract a magnetizable member inserted in or on a user's tongue to enhance the airflow.

### BRIEF DESCRIPTION OF THE DRAWINGS

FIG. 1 is a side view of the upper and lower inserts of the invention with the tension coil springs in place.

FIG. 2 is a side view of a coil spring having a protective plastic sleeve thereon used in the invention, and

FIG. 3 is an exploded view of the invention.

FIG. 4 is a side view of the raised portion for supporting the magnet of each of the inserts.

### DETAILED DESCRIPTION

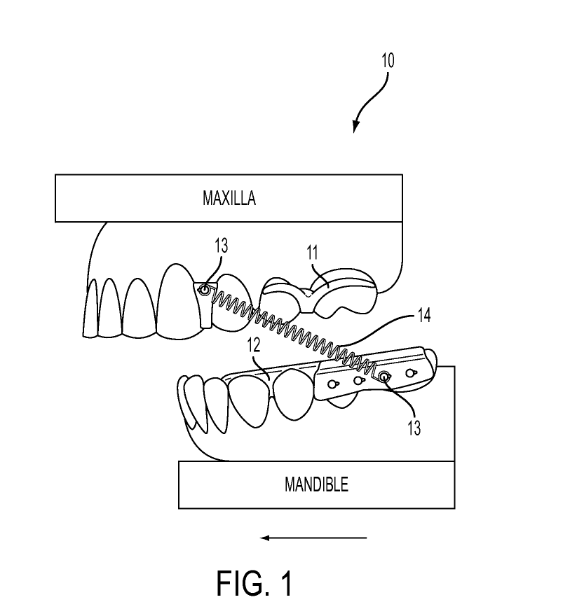

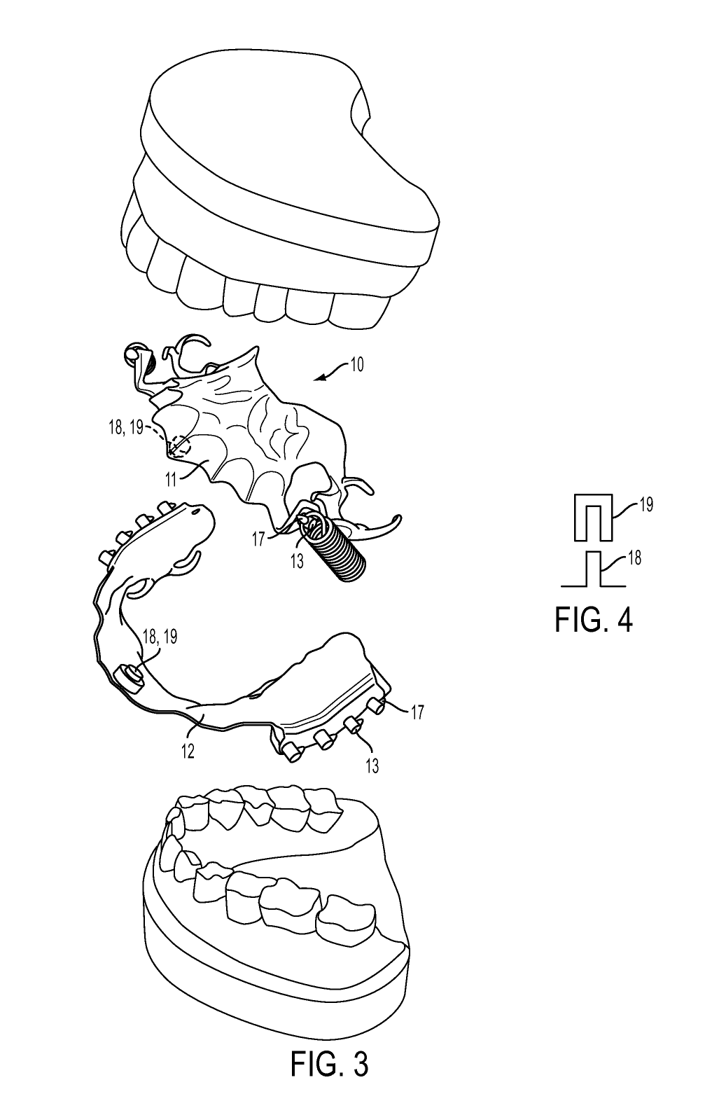

Referring to FIGS. 1 and 3 the appliance 10 includes an upper insert 11 that conforms with some of the user's upper teeth including maxillary dentition soft tissue and palate and includes a hook support 13 on each side of the insert, and a lower insert 12 that conforms to the user's lower teeth including mandibular dentition and soft tissue and that includes a plurality of hook supports 13 on each side of the insert. In one embodiment, there are four hook supports on each side of the lower insert 12. The upper hook support on each side of the upper insert is attached at a forward portion of the insert, and the plurality of hook supports on each side of the lower insert are attached at a rearward portion of the insert. The upper and lower inserts are made by casting using Vitallium® 2000, made by Dentsply Austenal, certified by Identalloy®. Other materials may be used, as well, which have similar properties to the cast alloy Vitallium® 2000, and is certified by Identalloy®. The upper and lower inserts may also be fabricated by a dental laboratory with the hook supports attached. The hook supports are cast with the upper and lower inserts in one unitary piece. Alternatively, the hook supports may be attached to the upper and lower inserts with an adhesive. The inserts 11, 12 may have arms 16 that extend from the inserts to conform to a plurality of teeth to keep the inserts in place in a user's mouth.

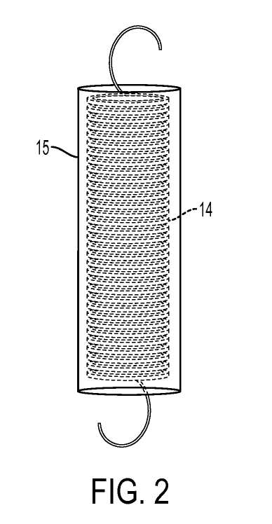

Each of the hook supports 13 are in the form of a smooth stem that fits into corresponding holes in the upper and lower trays and are bonded thereto or are integrally cast with the inserts. A pin 17 is integral with each stem and may be pointed and able to be bent to preclude detachment of the spring ends from their hook supports. The pins on the upper hook supports would point forwardly and those on the lower hook supports would point rearwardly as seen in the drawings. Each spring 14 shown in FIG. 2 may be made of plastic or stainless steel and has a curved hook at each end. A sleeve 15 as seen in FIG. 2 may be positioned over each spring to guard against injury to a user's gums. The sleeve 15 may be made of plastic or other suitable material.

The inserts 11, 12 have a raised portion 18 in the middle front portion of both the upper and lower inserts or at any other convenient location to attach a magnet 19 thereto. See FIG. 4 for a a side view of the raised portion 18 for supporting the magnet 19 of each of the inserts. The magnets are covered by a thin plastic or other material to prevent rusting. A member of the medical profession would surgically place a small piece of magnetizable material that the magnets would attract in the anterior portion of the tongue.

In use, a doctor would determine the proper strength coil springs to be used and would hook one end of the coil springs to the hook support on each side of the upper insert and hook the other end of the coil springs to one of the plurality of hook supports on each side of the lower insert. The particular hook support used to hook the spring ends on the lower tray would be determined by the doctor to provide a predetermined tension force to each side of the lower tray to advance the mandible forward under the proper tension in the direction of the arrow seen in FIG. 1. The user's tongue would also be positioned forwardly by the magnets to enhance air flow.

The appliance of the invention provides many advantages over prior art appliances including posterior support for some posterior teeth, complete vertical and lateral freedom of movement of the mandible, no sharp edges that may injure a user, and easy adjustment of the mandible advancement force. Furthermore, the tension springs are less likely to break as compared with elastic bands.

It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation

What is claimed is:

1. An oral appliance for use in advancing a user's lower jaw, comprising:
upper and lower inserts that fit to a user's upper and lower teeth,
a pair of hook supports attached one on each side of the forward portion of the upper insert,
a plurality of hook supports attached to each side of the rear portion of the lower insert, and
a pair of tension coil springs, each spring hooked at one end thereof to the hook support at each side of the upper insert, and hooked to one of the plurality of hook supports at each side of the lower insert, wherein the spring force of each coil spring on the lower tray may be adjusted by moving the hooked end on each side of the lower tray to another hook support, and
the upper and lower inserts have magnets attached thereto, each of the magnets being attractable to a magnetizable material on a user's tongue.

2. The oral appliance of claim 1, wherein each hook support comprises a stem extending therefrom, the stem fitting into a corresponding hole in the upper and lower trays.

3. The oral appliance of claim 1, further comprising a plastic sleeve positioned over each of the coil springs.

4. The oral appliance of claim 1, wherein the plurality of hook supports comprises four hook supports on each side of the lower insert.

5. The oral appliance of claim 1, wherein coil springs are made of stainless steel.

6. The oral appliance of claim 1, wherein the upper and lower inserts are made by casting using a dental casting alloy.

7. An oral appliance for use in advancing a user's lower jaw, comprising:
upper and lower inserts that fit to a user's upper and lower teeth respectively,
hook supports attached one on each side of the forward portion of the upper insert, and at least one hook support attached one on each side of the rearward portion of the lower insert,
a pair of tension devices attached between the upper and lower hook supports on both sides of the the upper and lower inserts to tend to pull the user's jaw forward,
and at least one magnet attached to at least one of the upper or lower inserts and attractable to a magnetizable material on or in a user's tongue to keep the user's tongue in an extended position.
